(19) United States
(12) Patent Application Publication
(10) Pub. No.: US 2017/0105913 A1
(43) Pub. Date: Apr. 20, 2017

(54) High Cleaning Silica With Low Abrasion And Method Of Making Same

(71) Applicant: J.M. Huber Corporation, Atlanta, GA (US)

(72) Inventor: Fitzgerald A. Sinclair, Smyrna, DE (US)

(73) Assignee: J.M. Huber Corporation

(21) Appl. No.: 15/346,786

(22) Filed: Nov. 9, 2016

Related U.S. Application Data
(62) Division of application No. 14/201,399, filed on Mar. 7, 2014.
(60) Provisional application No. 61/790,280, filed on Mar. 15, 2013.

(51) Int. Cl. A61K 8/25; A61Q 11/00; A61K 8/02; C01B 33/193

(52) U.S. Cl. CPC A61K 8/25; C01B 33/193; A61Q 11/00; A61K 8/0275; C01P 2004/51; C01P 2004/64; C01P 2006/12; C01P 2006/19; A61K 2800/28; A61K 2800/413; A61K 2800/805

(57) ABSTRACT

Silica materials having high cleaning and low abrasion properties are described, together with methods of making such materials and dentifrice compositions comprising the silica materials.

### CROSS-REFERENCE TO RELATED APPLICATIONS

This application claims priority to U.S. Provisional Application Ser. No. 61/790,280, filed Mar. 15, 2013, which is hereby incorporated by reference.

### BACKGROUND

Technical Field

The present disclosure relates to silica and silicate materials, and specifically to treated silica and metal silicate materials that can provide improved cleaning properties in a dentifrice composition.

Technical Background

Conventional dentifrice compositions comprise an abrasive substance to assist in the removal of dental deposits. One such dental deposit is pellicle, a protein film which adheres strongly to tooth surfaces and often contains brown or yellow materials that can result in tooth discoloration. A dentifrice should be sufficiently abrasive to clean the tooth surface, but not so abrasive as to damage the hard tissues of the tooth. In conventional dentifrice silica compositions, the abrasive properties of the silica composition typically increase as the cleaning properties increase. Accordingly, a silica composition that can provide desirable pellicle cleaning properties can exhibit undesirably high abrasive properties that can damage sensitive tooth tissues.

To date, conventional abrasive materials have limitations related to improving cleaning properties while maintaining desirable abrasive properties. Accordingly, there exists a general need to develop new dental abrasives and dentifrices thereof that exhibit high pellicle film cleaning properties and have acceptable abrasive properties. This need and other needs are satisfied by the compositions and methods of the present disclosure.

### SUMMARY

In accordance with the purpose(s) of the invention, as embodied and broadly described herein, this disclosure, in one aspect, relates to silica and silicate materials, and specifically to treated silica and metal silicate materials that can provide improved cleaning properties in a dentifrice.

In one aspect, the present disclosure provides a silica material comprising at least three of: a BET surface area of less than about 90 m²/g; an oil absorption number of at least about 80 cc/100 g; a loss on ignition of less than about 4 wt. %; and a PCR:RDA ratio of at least about 0.8 in a dentifrice comprising 11 wt. % glycerine (99.7%), 42.107 wt. % sorbitol (70%), 20 wt. % deionized water, 3 wt. % polyethylene glycol (PEG-12), 0.6 wt. % sodium carboxymethylcellulose, 0.5 wt. % tetrasodium pyrophosphate, 0.2 wt. % sodium saccharin, 0.243 wt. % sodium fluoride, 0.5 wt. % titanium dioxide, 1.2 wt. % sodium lauryl sulfate, 0.65 wt. % flavoring, and 20 wt. % of the silica material.

In another aspect, the present disclosure provides a dentifrice composition comprising the silica material described herein.

In another aspect, the present disclosure provides a method for preparing a silica material, the method comprising subjecting a precursor material to hydrothermal conditions to form an amorphous silica material.

In another aspect, the present disclosure provides a method for preparing a silica material, the method comprising: contacting a silicate compound or a solution thereof with an acid in the presence of a salt or solution thereof, so as to form a precursor material, and then subjecting the precursor material to hydrothermal conditions.

Additional aspects of the invention will be set forth in part in the description, examples, and figures which follow, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying figures, which are incorporated in and constitute a part of this specification, illustrate several aspects and together with the description serve to explain the principles of the invention.

FIG. 1 is a photo electron micrograph of a precursor material, prior to undergoing hydrothermal treatment, in accordance with various aspects of the present disclosure.

FIG. 2 is a photo electron micrograph of a silica material after undergoing hydrothermal treatment, in accordance with various aspects of the present disclosure.

FIG. 3 is a photograph micrograph of a crystalline silicate material, quartz, formed in a hydrothermal treatment process.

FIG. 4 is a scanning electron micrograph of a high cleaning silica material prepared in accordance with various aspects of the present disclosure.

FIG. 5 is a scanning electron micrograph of a high cleaning silica material prepared in accordance with various aspects of the present disclosure.

FIG. 6 is a scanning electron micrograph of a high cleaning silica material prepared in accordance with various aspects of the present disclosure.

FIG. 7 is a scanning electron micrograph of a high cleaning silica material prepared in accordance with various aspects of the present disclosure.

FIG. 8 is a scanning electron micrograph of a high cleaning silica material prepared in accordance with various aspects of the present disclosure.

FIG. 9 is a scanning electron micrograph of a high cleaning silica material prepared in accordance with various aspects of the present disclosure.

FIG. 10 is a graph of mercury porosimetry data (intrusion volume vs. pore diameter) for the inventive high cleaning silica and a conventional silica material, in accordance with various aspects of the present disclosure.

FIG. 11 is a schematic illustration of tightly packed particles that make up a conventional silica material.

FIG. 12 is a schematic illustration of the inventive silica material exhibiting larger void spaces between larger individual particles, in accordance with various aspects of the present disclosure.

FIG. 13 illustrates viscosity build data for the inventive silica material and conventional silica thickener materials, in accordance with various aspects of the present disclosure.

FIG. 14 is an x-ray diffraction (XRD) pattern from analysis of an inventive silica material, illustrating a lack of crystallinity.

FIG. 15 is an XRD pattern from analysis of a crystalline magadiite material to illustrate crystallinity.

FIG. 16 is an XRD pattern from analysis of a crystalline quartz material to illustrate crystallinity.

Additional aspects of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples and Figures included therein.

Before the present compounds, compositions, articles, systems, devices, and/or methods are disclosed and described, it is to be understood that they are not limited to specific synthetic methods unless otherwise specified, or to particular reagents unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, example methods and materials are now described.

As used herein, silica includes silicates. Silicates can include calcium silicate, magnesium silicate, aluminosilicates and sodium aluminosilicates. In various aspects, a silica and/or silicate material can comprise up to, for example, about 12% aluminum, calcium, sodium, or a combination thereof. In another aspect, a silica and/or silicate material does not comprise aluminum and/or calcium in quantities greater than trace or impurity levels.

As used herein, unless specifically stated to the contrary, the singular forms “a,” “an” and “the” include plural referents unless the context clearly dictates otherwise. Thus, for example, reference to “a filler” or “a solvent” includes mixtures of two or more fillers, or solvents, respectively.

As used herein, the terms “optional” or “optionally” means that the subsequently described event or circumstance can or cannot occur, and that the description includes instances where said event or circumstance occurs and instances where it does not.

For purposes of this invention, a “dentifrice” has the meaning defined in Oral Hygiene Products and Practice, Morton Pader, Consumer Science and Technology Series, Vol. 6, Marcel Dekker, N Y 1988, p. 200, which is incorporated herein by reference. Namely, a “dentifrice” is “ . . . a substance used with a toothbrush to clean the accessible surfaces of the teeth. Dentifrices are primarily composed of water, detergent, humectant, binder, flavoring agents, and a finely powdered abrasive as the principal ingredient . . . a dentifrice is considered to be an abrasive-containing dosage form for delivering anti-caries agents to the teeth.” Dentifrice formulations contain ingredients which should be dissolved prior to incorporation into the dentifrice formulation (e.g. anti-caries agents such as sodium fluoride, sodium phosphates, flavoring agents such as saccharin).

The Brass Einlehner (BE or BEA) Abrasion test used to measure the hardness of the silica materials reported in this application is described in detail in U.S. Pat. No. 6,616,916, incorporated herein by reference, involves an Einlehner AT-1000 Abrader generally used as follows: (1) a Fourdrinier brass wire screen is weighed and exposed to the action of a 10% aqueous silica suspension for a fixed length of time; (2) the amount of abrasion is then determined as milligrams brass lost from the Fourdrinier wire screen per 100,000 revolutions. The result, measured in units of mg loss, can be characterized as the 10% brass Einlehner (BE) abrasion value. Supplies useful for this test, for example, brass screens, wear plates, and tubing, are available from Duncan Associates, Rutland, Vt., USA. In an exemplary BEA measurements, brass screens, such as Phosphos Bronze P.M., can be prepared by washing in hot, soapy water (0.5% Alconox) in an ultrasonic bath for 5 minutes, then rinsed in tap water and rinsed again in a beaker containing 150 ml water set in an ultrasonic bath. The screen can be rinsed again in tap water, dried in an oven set at 105° C. for about 20 minutes, cooled in a dessicator, and weighed. Screens can be handled using tweezers to prevent contamination from skin oils. An Einlehner test cylinder can be assembled with a wear plate and weighed screen (e.g., red line side down—not abraded side) and clamped in place. A wear plate can typically be used for about 25 tests, whereas a weighed screen is typically used once. A 10 wt. % silica slurry can be prepared by mixing 100 g of silica with 900 g of deionized water, and be poured into the Einlehner test cylinder. Einlehner PVC tubing can be placed onto the agitating shaft. The tubing typically has 5 numbered positions. For each test, the position of the tubing can be incrementally adjusted until it has been used five times. The Einlehner abrasion instrument can be re-assembled and set to run for 87,000 revolutions. Each test typically takes about 49 minutes. When the cycle is completed, the screen can be removed, rinsed in tap water, placed in a beaker containing water, and set in an ultrasonic bath for 2 minutes, rinsed with deionized water and dried in an oven set at 105° C. for 20 minutes. The dried screen can be cooled in a dessicator and reweighed. Two tests are typically run for each sample and the results are averaged and expressed in mg lost per 100,000 revolutions.

The Radioactive Dentin Abrasion (RDA) values of dentifrices containing the silica compositions used in this invention are determined according to the method set forth by Hefferen, Journal of Dental Res., July-August 1976, 55 (4), pp. 563-573, and described in Wason U.S. Pat. Nos. 4,340,583, 4,420,312 and 4,421,527, which publications and patents are incorporated herein by reference. An exemplary RDA test method comprises the following steps:

A. Selection and preparation of teeth—Sound, single-rooted permanent teeth that are caries-free and vital at extraction can be selected. Teeth can then be scraped clean with a scalpel. The crown and root tip of each tooth can be removed using an abrasive disc so as to prepare a dentin sample 14 mm long and at least 2 mm wide at the narrower end. Cut pieces of root (dentin chips) or, alternatively, an additional tooth, can also be prepared to be later used in determining a correction factor for self-absorption of radiation.

B. Irradiation of dentin—The prepared roots and dentin chips described in Step A can be exposed to a neutron flux of 2×10¹² neutrons/cm² for three hours.

C. Mounting of roots—After irradiation, the irradiated roots can be embedded in a mount of cold-ring dental methacrylate resin and mounted onto a cross-brushing machine. Toothbrushes used throughout the test can be 50-Tuft, medium, flat, “Pepsodent” toothbrushes.

D. Preconditioning the dentin surfaces—Prior to initial test run, the freshly mounted, irradiated roots can be brushed with a reference slurry (10 g calcium pyrophosphate+50 ml of a 0.5% CMC-10% glycerine solution) for 6,000 brush strokes. At the beginning of each subsequent day's test run, the roots can be brushed for 1,000 strokes.

E. Test run—After preconditioning, the dentin samples can be conditioned with the reference slurry (same slurry as in Step D) for 1,500 brush strokes at the beginning, during and after each test run. The test run can consist of brushing dentin samples for 1,500 brush strokes with a slurry of test product (25 g dentifrice+40 ml deionized of distilled water).

F. Preparation of correction factors—The correction factors can be prepared by dissolving the dentin chips or, alternatively, an additional tooth, from Step B in 5 ml. conc. HCl brought to a volume of 250 ml. with distilled water. One ml. of this solution can be added to test pastes and reference slurries which can be prepared similarly to those in Step E, and then neutralized with 0.1 N NaOH.

Radioactive Tracer Counting—The radioactivity of the slurry samples (1.0 ml.) can be determined with an Intertechnique SL-30 liquid scintillation counter. Alternate counting procedure: 3 ml. aliquots of each slurry can be transferred to stainless steel, flat-bottom 1 inch.times. 5/16 inch planchets and counted using Nuclear Chicago Geiger Counting System.

Calculations—The radioactive dentin abrasion value (RDA) for a particular paste will be the ratio of the average corrected counts for that paste to the average count for the reference multiplied by 100. The reference abrasive is given an arbitrary dentin abrasion value of 100 units.

The cleaning property of dentifrice compositions is typically expressed in terms of Pellicle Cleaning Ratio (“PCR”) value. The PCR test measures the ability of a dentifrice composition to remove pellicle film from a tooth under fixed brushing conditions. The PCR test is described in “In Vitro Removal of Stain with Dentifrice” G. K. Stookey, et al., J. Dental Res., 61, 1236-9, 1982. Both PCR and RDA results vary depending upon the nature and concentration of the components of the dentifrice composition. PCR and RDA values are unitless.

In an exemplary RDA test, 8 mm square bovine enamel blocks can be mounted in methacrylate blocks sized to fit into a V-8 brushing machine. Specimens can be wet polished with 600 grit SiC paper followed by flour of pumice and then be sonicated to remove debris. Ground specimens can be etched in 1% HC (60 sec.) followed by saturated NaCO₃ solution. A final immersion in 1% phytic acid solution can clean off remaining debris. Specimens can be mounted in humidified and heated staining wheels as also described by Stookey and subjected to a stain procedure including immersion of specimens in 5.4% TSB staining broth comprising 2.5% mucin, 50 ppm ferric chloride, 0.338% instant coffee (Folgers, Procter and Gamble Inc.), 0.338% instant tea Liptons—non sweetened, Liptons, Inc.) and 4 innoculums of Sarcina Lutea bacterial culture. The staining solution can be applied for a period ranging from 4-8 days, with specimens withdrawn based upon Chromameter assessment of color. Stained specimens can be evaluated for tooth color utilizing a Chromameter. Tooth specimens can be qualified with 25<L<40 (L indicates the light/dark assessment in conventional CIELAB color space) as assessed on the chromameter. A special sample jig can be prepared to allow pre and post brushing color valuations. Initial color assessments can allow teeth to be sorted and rank ordered for treatment distribution normalized to tooth color. Specimens can be stratified to treatment groups based upon initial L values for each treatment group. Toothbrushing can be carried out in a V8 brushing machine using, for example, Oral B® 40 toothbrushes (Oral B 40 Regular Toothbrush, Oral B Inc., Belmont, Calif.) pre-conditioned at 20,000 strokes at a 150 gram normalized load. One treatment can include a control reference standard prepared by adding 10 grams calcium pyrophosphate (ADA reference standard calcium pyrophosphate, Monsanto Inc., St. Louis, Mo.) to 50 grams of 5.2 CMC (carboxymethylcellulose) solution. Dentifrices can be applied in 25/40 dentifrice/water slurries prepared with a biohomogenizer wand. Treatments can also be rotated so that enamel specimens from each treatment group are brushed in each position on the V8 brushing machine. Specimens can be brushed in test slurries for 800 strokes at a normalized pressure of 150 grams. Following brushing, specimens can be air dried and re-examined for color L values on the Chromameter. Treatment effects can then be assessed according relative efficacy as compared against the reference calcium pyrophosphate abrasive as follows:

(L_{tf}−Lₜᵢ)/(L_{cf}−L_{ci})*100=PCR

where Lₜᵢ and L_{tf} are L chromameter values for test dentifrice treated specimens initial and post brushing and L_{ci} and L_{cf} are values for calcium pyrophosphate abrasive control respectively. For statistical analysis, the raw cleaning score for each tooth, L_{tf}−Lₜᵢ within each treatment group can be calculated. These individual scores can then be converted to cleaning ratio scores (PCR scores) by division of these individual scores by the average raw cleaning score obtained for calcium pyrophosphate ADA abrasive control and multiplication by 100.

Dentifrice compositions for performing PCR and/or RDA testing can vary. In one aspect, a dentifrice composition for PCR and/or RDA testing can comprise: 11 wt. % glycerine (e.g, 99.7%), 42.107 wt. % sorbitol (e.g., 70%), 20 wt. % deionized water, 3 wt. % polyethylene glycol (e.g., PEG-12), 0.6 wt. % sodium carboxymethylcellulose (e.g., Cekol 2000, available from CP Kelco US, Inc.), 0.5 wt. % tetrasodium pyrophosphate, 0.2 wt. % sodium saccharin, 0.243 wt. % sodium fluoride, 0.5 wt. % titanium dioxide, 1.2 wt. % sodium lauryl sulfate, 0.65 wt. % flavoring, and 20 wt. % of the silica material.

The surface area of a silica material can be determined using conventional surface area analysis techniques, such as, for example, Brunauer, Emmett, and Teller (“BET”) and cetyltrimethylammonium bromide (“CTAB”). BET surface area measurements are determined by measuring the amount of nitrogen adsorbed on a surface, as described in Brunaur et al., J. Am. Chem. Soc., 60, 309 (1938). CTAB surface area measurements are determined by measuring the adsorption of a large molecule, cetyltrimethylammonium bromide, on a surface. In one aspect, a given weight of silica material is combined with a quantity of cetyltrimethylammonium bromide, and the excess separated by centrifuge and quantitated by titration with sodium lauryl sulfate using a surfactant electrode. The external surface of the silica material can be determined from the quantity of CTAB adsorbed (analysis of CTAB before and after adsorption). In a specific example, about 0.5 g of silica is placed in a 250-ml beaker with 100.00 ml CTAB solution (5.5 g/L), mixed on an electric stir plate for 1 hour, then centrifuged for 30 minutes at 10,000 rpm. One ml of 10% Triton X-100 is added to 5 ml of the clear supernatant in a 100-ml beaker. The pH is adjusted to 3.0-3.5 with 0.1 N HCl and the specimen is titrated with 0.0100 M sodium lauryl sulfate using a surfactant electrode (Brinkmann SUR1501-DL) to determine the endpoint.

Loss on ignition (“LOI”) values refer to the weight lost upon heating a silica material at 900° C. for 2 hours, after pre-drying the silica material for 2 hours at 105° C. LOI is a measure of hydroxyl group content of a silica material.

Oil Absorption, frequently expressed as the oil absorption number (“OAN”), of a silica material can be determined using a rubout method, wherein a quantity of linseed oil is mixed with a silica material by rubbing with a spatula on a smooth surface until a stiff putty-like paste is formed. The amount of oil needed to form a stiff paste that curls when spread out is measured. The OAN can then be expressed as the volume of oil required per unit weight of silica material to saturate the silica material. A higher oil absorption level indicates a higher structure silica, for example, aggregates having a higher amount of void space between primary individual fused silica particles. Similarly, a low oil absorption value indicates a low structure silica, for example, aggregates having a smaller amount of void space between primary individual fused silica particles. In one aspect, linseed oil can be used to determine the oil absorption value for a silica material.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

Each of the materials disclosed herein are either commercially available and/or the methods for the production thereof are known to those of skill in the art.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As briefly described above, the present disclosure provides a silica composition that can be useful in, for example, a dentifrice composition. In one aspect, the inventive silica composition can provide high cleaning properties while maintaining desirable abrasive properties. In other aspects, the present disclosure provides methods for preparing the inventive silica compositions and dentifrice compositions comprising the inventive silica compositions.

In the oral care industry, it would be desirable to have dentifrice materials with improved cleaning properties. It would also be advantageous for such dentifrice materials to exhibit moderate dentin and enamel abrasion properties, so as to not damage teeth during repeated use.

### Properties of High Cleaning Silica

In various aspects, the inventive high cleaning silica can exhibit a low surface area; a high oil absorption number; a low loss on ignition; a PCR:RDA ratio of about 0.8 or more, or about 1 or more in a dentifrice composition at 20 wt. % loading; a low Einlehner abrasion; or a combination thereof. In another aspect, the high cleaning silica can be amorphous or at least partially amorphous, as determined by X-ray diffraction measurements.

In other aspects, the inventive high cleaning silica can comprise an agglomeration of dense, spherical or partially spherical particles. In yet another aspect, the inventive silica can comprise an agglomeration of dense particles each having a diameter of about 80 nm. In yet another aspect, the inventive silica can comprise agglomerates of individual, fused silica particles spaced so as to exhibit desirable interstitial pores between the individual particles and provide high oil absorption numbers.

The inventive high cleaning silica material can exhibit PCR values, when used in a dentifrice at 20% loading, of from about 50 to about 150, for example, 50, 52, 54, 56, 58, 60, 62, 64, 66, 68, 70, 72, 74, 76, 78, 80, 82, 84, 86, 88, 90, 92, 94, 96, 98, 100, 102, 10, 106, 108, 110, 112, 114, 116, 118, 120, 122, 124, 126, 128, 130, 132, 134, 136, 138, 140, 142, 144, 146, 148, or 150; from about 75 to about 150, for example, about 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, or 150; or from about 80 to about 125, for example, 80, 82, 84, 86, 88, 90, 92, 94, 96, 98, 100, 102, 104, 106, 108, 110, 112, 114, 116, 118, 120, 122, 124, or 125. In other aspects, the inventive high cleaning silica can exhibit PCR values of less than about 50 or greater than about 180 when used in a dentifrice at 20 wt. % loading, and the present invention is not intended to be limited to any particular PCR value.

The inventive high cleaning silica material can exhibit RDA values, when used in a dentifrice at 20% loading, of from about 50 to about 150, for example, 50, 52, 54, 56, 58, 60, 62, 64, 66, 68, 70, 72, 74, 76, 78, 80, 82, 84, 86, 88, 90, 92, 94, 96, 98, 100, 102, 10, 106, 108, 110, 112, 114, 116, 118, 120, 122, 124, 126, 128, 130, 132, 134, 136, 138, 140, 142, 144, 146, 148, or 150; from about 55 to about 125, for example, about 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, or 125; or from about 60 to about 125, for example, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, or 125. In other aspects, the inventive high cleaning silica can exhibit RDA values of less than about 50 or greater than about 150 when used in a dentifrice at 20 wt. % loading, and the present invention is not intended to be limited to any particular RDA value.

In conventional silica materials that can be used in dentifrice applications, an increase in PCR value results in a corresponding RDA value. Such increases in RDA can result in undesirably high abrasion properties that can damage tooth tissues. In one aspect, the high cleaning silica of the present invention exhibits decoupled PCR and RDA values, such that a high PCR can be obtained while maintaining a desirable RDA value.

In one aspect, the high cleaning silica can exhibit a PCR:RDA ratio of at least about 0.8, for example, about 0.85, 0.87, 0.91, 0.93, 0.95, 0.97, 0.99, 1, 1.01, 1.03, 1.05, 1.07, 1.09, 1.1, 1.2, 1.3, 1.4, 1.5, or greater when used in a dentifrice at 20 wt. % loading. In other aspects, the high cleaning silica can exhibit a PCR:RDA ratio of from about 0.85 to about 1.5, for example, about 0.85, 0.86, 0.88, 0.9, 0.92, 0.94, 0.96, 0.98, 1, 1.1, 1.2, 1.3, 1.4, or 1.5; from about 0.9 to about 1.5, for example, about 0.9, 0.92, 0.94, 0.96, 0.98, 1, 1.1, 1.2, 1.3, 1.4, or 1.5; from about 0.95 to about 1.5, for example, about 0.95, 0.96, 0.98, 1, 1.1, 1.2, 1.3, 1.4, or 1.5; from about 0.98 to about 1.5, for example, about 0.98, 1, 1.1, 1.2, 1.3, 1.4, or 1.5; or from about 1 to about 1.5, for example, about 1, 1.1, 1.2, 1.3, 1.4, or 1.5 when used in a dentifrice at 20 wt. % loading. In other aspects, the PCR:RDA ratio can be greater than about 1.5, for example, about 1.6, 1.7, 1.8, 1.9, 2, 2.1, 2.2, 2.3, 2.4, 2.5, 3, 3.5, 4, or more when used in a dentifrice at 20 wt. % loading.

In one aspect, the inventive high cleaning silica can exhibit a BET surface area of less than about 140 m²/g, for example, about 5, 10, 15, 20, 25, 30, 35, 40, 45, 50, 55, 60, 65, 70, 75, 80, 85, 90, 95, 100, 110, 120, 130, or 140 m²/g; less than about 100 m²/g, for example, about 5, 10, 15, 20, 25, 30, 35, 40, 45, 50, 55, 60, 65, 70, 75, 80, 85, 90, 95, or 100 m²/g; less than about 90 m²/g, for example, about 5, 10, 15, 20, 25, 30, 35, 40, 45, 50, 55, 60, 65, 70, 75, 80, 85, or 90 m²/g; less than about 75 m²/g, for example, about 5, 10, 15, 20, 25, 30, 35, 40, 45, 50, 55, 60, 65, 70, or 75 m²/g; less than about 60 m²/g, for example, about 5, 10, 15, 20, 25, 30, 35, 40, 45, 50, 55, or 60 m²/g; less than about 55 m²/g, for example, about 5, 10, 15, 20, 25, 30, 35, 40, 45, 50, or 55 m²/g; less than about 50 m²/g, for example, about 5, 10, 15, 20, 25, 30, 35, 40, 45, or 50 m²/g; or less than about 45 m²/g, for example, about 5, 15, 20, 25, 30, 35, 40, or 45 m²/g. In another aspect, the high cleaning silica can exhibit a BET surface area of from about 5 m²/g to about 60 m²/g, from about 5 m²/g to about 55 m²/g, from about 5 m²/g to about 50 m²/g, or from about 5 m²/g to about 45 m²/g. In another aspect, the high cleaning silica can exhibit a BET surface area of from about 3 m²/g to about 140 m²/g, from about 3 m²/g to about 100 m²/g, from about 3 m²/g to about 75 m²/g, or from about 3 m²/g to about 60 m²/g. In still other aspects, the high cleaning silica can exhibit a BET surface area of less than about 15 m²/g or for thickener BET greater than about 75 m²/g, and the present invention is not intended to be limited to any particular BET surface area.

The high cleaning silica can also exhibit an oil absorption number of greater than about 80 cc/100 g, for example, about 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200 cc/100 g, or more; greater than about 90 cc/100 g, for example, about 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200 cc/100 g, or more; greater than about 110 cc/100 g; greater than about 120 cc/100 g; greater than about 130 cc/100 g, greater than about 140 cc/100 g, greater than about 150 cc/100 g, greater than about 160 cc/100 g, greater than about 170 cc/100 g, or greater than about 180 cc/100 g. In another aspect, the high cleaning silica can exhibit an oil absorption number of from about 90 cc/100 g to about 200 cc/100 g, from about 100 cc/100 g to about 200 cc/100 g, from about 110 cc/100 g to about 200 cc/100 g, from about 120 cc/100 g to about 200 cc/100 g, from about 130 cc/100 g to about 200 cc/100 g, from about 140 cc/100 g to about 200 cc/100 g, from about 150 cc/100 g to about 200 cc/100 g, from about 160 cc/100 g to about 200 cc/100 g, from about 170 cc/100 g to about 200 cc/100 g, or from about 180 cc/100 g to about 200 cc/100 g. In other aspects, the high cleaning silica can exhibit an oil absorption number greater than about 200 cc/100 g, and the present invention is not intended to be limited to any particular oil absorption number.

In one aspect, the high cleaning silica can exhibit a loss on ignition of less than about 5 wt. %. In another aspect, the high cleaning silica can exhibit a loss on ignition of less than about 4 wt. %, for example, about 0.1, 0.2, 0.3, 0.4, 0.5, 0.7, 0.9, 1, 1.1, 1.3, 1.5, 1.7, 1.9, 2.1, 2.3, 2.5, 2.7, 2.9, 3.1, 3.3, 3.5, 3.7, 3.9, or 4 wt. %; of less than about 3.5 wt. %, less than about 3 wt. %, less than about 2.5 wt. %, or less than about 2 wt. %. In other aspects, the high cleaning silica can exhibit a loss on ignition of from about 0.5 wt. % to about 4 wt. %, for example, about 0.5, 0.7, 0.9, 1, 1.1, 1.3, 1.5, 1.7, 1.9, 2.1, 2.3, 2.5, 2.7, 2.9, 3.1, 3.3, 3.5, 3.7, 3.9, or 4 wt. %; from about 0.5 wt. % to about 3.5 wt. %; from about 0.5 wt. % to about 3 wt. %; from about 0.5 wt. % to about 2.5 wt. %; or from about 0.3 wt. % to about 2 wt. %.

In yet another aspect, the high cleaning silica can exhibit a BEA from about 0.7 to about 21 mg, for example, about 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 13, 14, 15, 16, 17, 18, 19, 20 or 21 mg.; or from about 0.7 to about 13 mg, for example, about 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, or 13 mg. In other aspects, the high cleaning silica can exhibit a BEA of less than about 0.7 mg or greater than about 21 mg, and the present invention is not intended to be limited to any particular BEA range or value.

The particle size of the high cleaning silica can vary, depending upon the specific method of preparation. In various aspects, the average particle size of the high cleaning silica can range from about 8 μm to about 30 μm, for example, about 8, 9, 10, 11, 12, 13, 14, 15, 16, 17, 18, 19, 20, 21, 22, 23, 24, 25, 26, 27, 28, 29, or 30 μm. In other aspects, the average particle size of the high cleaning silica can range from about 8 μm to about 25 μm, for example, about 8, 9, 10, 11, 12, 13, 14, 15, 16, 17, 18, 19, 20, 21, 22, 23, 24, or 25 μm; from about 10 μm to about 20 μm, for example, about 10, 11, 12, 13, 14, 15, 16, 17, 18, 19, or 20 μm; or from about 12 μm to about 19 μm, for example, about 12, 13, 14, 15, 16, 17, 18, or 19 μm. In still other aspects, the average particle size can be less than about 8 μm or greater than about 30 μm, and the present invention is not intended to be limited to any particular particle size. It should also be noted that particle size of silica materials is a distributional property and that the average particle size and distribution of particle sizes in a given sample can vary depending on, for example, the sampling conditions.

In one aspect, the high cleaning silica can exhibit any two, three, or four of a BET surface area of less than about 50 m²/g, an oil absorption number of at least about 110 cc/100 g, a loss on ignition of less than about 4 wt. %, a PCR:RDA ratio of at least about 0.9, or a combination thereof. In another aspect, the high cleaning silica can exhibit any two, three, or four of a BET surface area of less than about 45 m²/g, an oil absorption number of at least about 120 cc/100 g, a loss on ignition of less than about 3.5 wt. %, a PCR:RDA ratio of at least about 1, or a combination thereof. In one aspect, the combination of low surface area and high oil absorption number can provide agglomerates of large, dense silica particles having larger interstitial void spaces. In an exemplary aspect, the inventive high cleaning silica can exhibit a surface area lower than a conventional silica, such as, for example, ZEODENT® 103, available from J. M. Huber, while exhibiting an oil absorption number several times higher than the conventional silica. It should be noted that ZEODENT® 103, a conventional silica material can have PCR values of from about 100 to about 103, RDA values of from about 188 to about 220, and a PCR:RDA ratio of from about 0.45 to about 0.56.

In another aspect, the high cleaning silica can exhibit a desirable high brightness value. In other aspects, the high cleaning silica can exhibit a Technidyne brightness of at least about 80, at least about 85, at least about 90, at least about 95, or at least about 100.

When used in a dentifrice composition, the high structure of the inventive high cleaning silica material can also provide desirable thickening properties, and can reduce and/or eliminate the need for additional thickener materials in the dentifrice composition. Silica materials prepared as described herein can exhibit thickening properties equivalent to those of ZEODENT® 153, an industry standard dentifrice thickener, available from J.M. Huber. In one aspect, the inventive high cleaning silica materials can be useful in combination with cetylpyridinium chloride (“CPC”) formulations. In another aspect, the inventive high cleaning silica materials can be useful as an abrasive having increased flavor compatibility. In yet another aspect, the inventive high cleaning silica materials can be used in combination with other moderate or low cleaning silica materials to provide improved or boosted cleaning properties.

In yet another aspect, the high cleaning silica can exhibit a hydroxyl group density of less than about 2%, for example, about 0.5, 0.75, 1, 1.25, 1.5, 1.75, or 1.99%.

In one aspect, the high cleaning silica can exhibit a BET of less than about 75 m2/g, an oil absorption number of at least about 80 cc/100 g, a loss on ignition of less than about 4 wt. %, and a PCR:RDA ratio of at least about 0.8 when used in a dentifrice at 20% loading. In another aspect, the high cleaning silica can exhibit a BET of less than about 60 m2/g, an oil absorption number of from about 100 to about 200 cc/100 g, a loss on ignition of less than about 4 wt. %, and a PCR:RDA ratio of at least about 0.9 when used in a dentifrice at 20% loading. In another aspect, the high cleaning silica can exhibit a BET of less than about 60 m2/g, an oil absorption number of at least about 110 cc/100 g, a loss on ignition of less than about 4 wt. %, and a PCR:RDA ratio of at least about 0.9 when used in a dentifrice at a 20% loading. In still another aspect, the high cleaning silica can exhibit a BET of less than about 40 m2/g, an oil absorption number of from about 110 to about 200 cc/100 g, a loss on ignition of less than about 2 wt. %, and a PCR:RDA ratio of from about 0.8 to about 1.5 when used in a dentifrice at 20% loading. In still another aspect, the high cleaning silica can exhibit a BET of less than about 40 m2/g, an oil absorption number of from about 110 to about 200 cc/100 g, a loss on ignition of less than about 2 wt. %, a PCR of at least about 100 and a PCR:RDA ratio of from about 0.8 to about 1.5 when used in a dentifrice at 20% loading. In another aspect, the high cleaning silica can exhibit a BET of less than about 40 m2/g, an oil absorption number of from about 120 to about 200 cc/100 g, a loss on ignition of less than about 3 wt. %, and a PCR:RDA ratio of at least about 0.8 when used in a dentifrice at 20% loading. In yet another aspect, the high cleaning silica can exhibit a BET of less than about 40 m2/g, an oil absorption number of at least about 150 cc/100 g, a loss on ignition of less than about 2.5 wt. %, and a PCR:RDA ratio of at least about 0.8 when used in a dentifrice at 20% loading. In another aspect, the high cleaning silica can exhibit a BET of less than about 60 m2/g, an oil absorption number of from about 120 to about 200 cc/100 g, a loss on ignition of less than about 5 wt. %, and a PCR:RDA ratio of at least about 1.3 when used in a dentifrice at 20% loading. In another aspect, the high cleaning silica can exhibit a BET of less than about 60 m2/g, an oil absorption number of at least about 150 cc/100 g, a loss on ignition of less than about 5 wt. %, and a PCR:RDA ratio of at least about 1.25 when used in a dentifrice at 20% loading. In another aspect, the high cleaning silica can exhibit a BET of less than about 60 m2/g, an oil absorption number of from about 120 to about 200 cc/100 g, a loss on ignition of less than about 5 wt. %, a PCR of at least about 80 and a PCR:RDA ratio of at least about 1.3 when used in a dentifrice at 20% loading. In another aspect, the high cleaning silica can exhibit an oil absorption number of at least about 80 cc/100 g, a PCR of at least about 80 and a PCR:RDA ratio of at least about 0.8 when used in a dentifrice at 20% loading. In still another aspect, the high cleaning silica can exhibit an oil absorption number of at least about 100 cc/100 g, a PCR of at least about 80 and a PCR:RDA ratio of at least about 0.9 when used in a dentifrice at 20% loading.

### Preparation of High Cleaning Silica

Silica materials suitable for use in dentifrice compositions can comprise synthetically produced, precipitated silicas. In one aspect, the silica material can be a high structure silica material. These silica materials can be produced using various procedures. In one aspect, a precursor material can be subjected to hydrothermal conditions. In one aspect, a precursor material can be prepared by contacting a silicate compound, such as, for example, sodium silicate, with an acid to form a silicate solution in, for example, the presence of a salt or solution thereof. The silicate solution can then be combined with sulfuric acid and amorphous silica particles can be precipitated. In various aspects, such a precursor material can exhibit properties of a silica gel, a precipitated silica, or a combination thereof. In another aspect, a precursor material can comprise a precipitated silica and/or a silica gel.

The silicate compound can comprise any silicate compound suitable for use in preparing a silica material. In various aspects, any suitable alkali metal silicate can be used with the methods described herein, including metal silicates, disilicates, and the like. In one aspect a water soluble silicate, such as, for example, a potassium silicate, a sodium silicate, or a combination thereof, can be used. In one aspect, sodium silicate can be used. In other aspects, a silicate compound having a desirable metal:silicate molar ratio (MR) can be selected. For example, sodium silicates can generally have a metal:silicate molar ratio of from about 1:1 to about 1:3.5. In one aspect, the silicate compound can have a molar ratio of from about 1:1 to about 1:3.5, for example, about 1:1, 1:1.25, 1:1.5, 1:1.75, 1:2, 1:2.25; 1:2.5; 1:2.75; 1:3, 1:3.25, or 1:3.5; or from about 1:2.5 to about 1:3.5, for example, about 1:2.5; 1:2.75; 1:3, 1:3.25, or 1:3.5. In another aspect, the silicate compound can have a molar ratio of about 1:3.3.

In one aspect, one or more salts or solutions thereof can be added to a reaction vessel prior to precipitation of a silica material. In one aspect, a salt solution is added prior to the introduction of the silicate compound or solution thereof. In various aspects, the salt can comprise any one or more salts or solutions thereof that are compatible with the silicate and acid. In one aspect, the salt comprises a sulfate, such as, for example, sodium sulfate. In another aspect, the salt comprises a phosphate salt. In yet another aspect, the sale comprises a chloride. The concentration of salt utilized can vary, depending upon, for example, the specific reactants and conditions used for a particular process. In one aspect, the amount of salt used can range from about 0 g to about 30 g, for example, about 0, 0.5, 1, 1.5, 2, 3, 4, 5, 6, 7, 8, 10, 12, 14, 16, 18, 20, 22, 24, 26, 28, or 30 g, of salt per 100 g of water. In other aspects, the amount of salt used can be less than or greater than any particular value recited herein and the present invention is not intended to be limited to any particular salt concentration.

While not wishing to be bound by theory, it is believed that the presence of one or more salts during the precipitation and subsequent processes can affect the degree of crystallinity in a resulting silica material. In other aspects, the particular salt or anion selected can affect the crystallinity of a resulting silica material. In one aspect, a 10% solution of sodium sulfate can be used.

In one aspect, the silicate compound, such as, for example, sodium silicate, can be contacted with an acid to produce a silicate solution. In general, any acid capable of at least partially reacting with the silicate compound and forming a silicate solution can be used. In another aspect, the selection of a particular acid can vary, depending upon the specific silicate compound being used. In various aspects, the acid can comprise nitric acid, hydrochloric acid, phosphoric acid, boric acid, hydrofluoric acid, sulfuric acid, or a combination thereof. In other aspects, other suitable acids can be utilized in addition to or in lieu of any acid specifically recited herein. The silicate compound and acid can be contacted in any suitable ratio so as to provide a solution having a desirable silicate concentration. In one aspect, the solution comprises from about 8 wt. % to about 35 wt. % silicate, for example, about 8, 9, 11, 13, 15, 17, 19, 21, 23, 25, 27, 29, 31, 33, or 35 wt. % silicate. In another aspect, the solution comprises from about 8 wt. % to about 20 wt. % silicate, for example, about 8, 10, 12, 14, 16, 18, or 20 wt. % silicate. In other aspects, the resulting silicate solution can have a silicate concentration less than or greater than any value specifically recited herein, and the present disclosure is intended to cover such solutions. In still other aspects, silicate solutions are commercially available and can be purchased and utilized as-is (e.g., from Sigma-Aldrich Corporation, St. Louis, Mo., USA).

In one aspect, the acid can be initially added, while stirring, until the solution has a pH of about 8.0. In one aspect, the rate of acid addition can vary, provided that gelation is avoided until after precipitation begins. As acid is added and precipitation begins, the solution can exhibit opalescence and can begin to gel. Additional water can be added, if needed, to maintain a desired viscosity and stir speed.

Once formation of the precursor material is complete, the resulting precursor material can be subjected to a hydrothermal treatment at an elevated temperature and elevated pressure. As used herein, the term “hydrothermal” is intended to refer to a treatment or process wherein a sample is subjected to an elevated temperature and pressure in a closed and/or sealed environment. In one aspect, the precursor material can be disposed in a sealed reaction vessel and heated to a temperature of from about 160° C. to about 220° C. for a period of about 1.5 to about 2 hours. In other aspects, the precursor material can be disposed in a sealed reaction vessel and heated to a temperature of less than about 160° C. or greater than about 220° C., and the present invention is not intended to be limited to any particular heating temperature. It should be understood that heating at a lower temperature can be performed at a longer period of time to achieve a similar desired result. In various aspects, the elevated temperature of a hydrothermal process can range from about 100° C. to about 350° C., for example, about 100, 125, 150, 175, 200, 225, 250, 275, 300, 325, or 350° C.; from about 120° C. to about 240° C., for example, about 120, 140, 160, 180, 200, 220, or 240° C.; or from about 160° C. to about 220° C., for example, about 160, 170, 180, 190, 200, 210, or 220° C.

The elevated pressure of a hydrothermal process can be introduced via a separate source, such as, for example, a compressed gas supply, or from the autogenous pressure resulting from heating the sample in a sealed vessel. In another aspect, the precursor material can be stirred at, for example, about 400 rpm while heating. In another aspect, the precursor material is not stirred or otherwise agitated during the hydrothermal process. As described above with respect to time and temperature, it should be understood that one or more of the temperature, time, and pressure of a hydrothermal process can be adjusted to achieve a desired result. In one aspect, a hydrothermal process at a higher temperature and/or pressure can achieve the same results in less time, as compared to a similar process at a lower temperature and/or pressure. The vessel in which a hydrothermal process is conducted can comprise any materials and/or design suitable for use in such a process. In one aspect, the vessel comprises a sealable stainless steel container capable of withstanding elevated temperature and pressure.

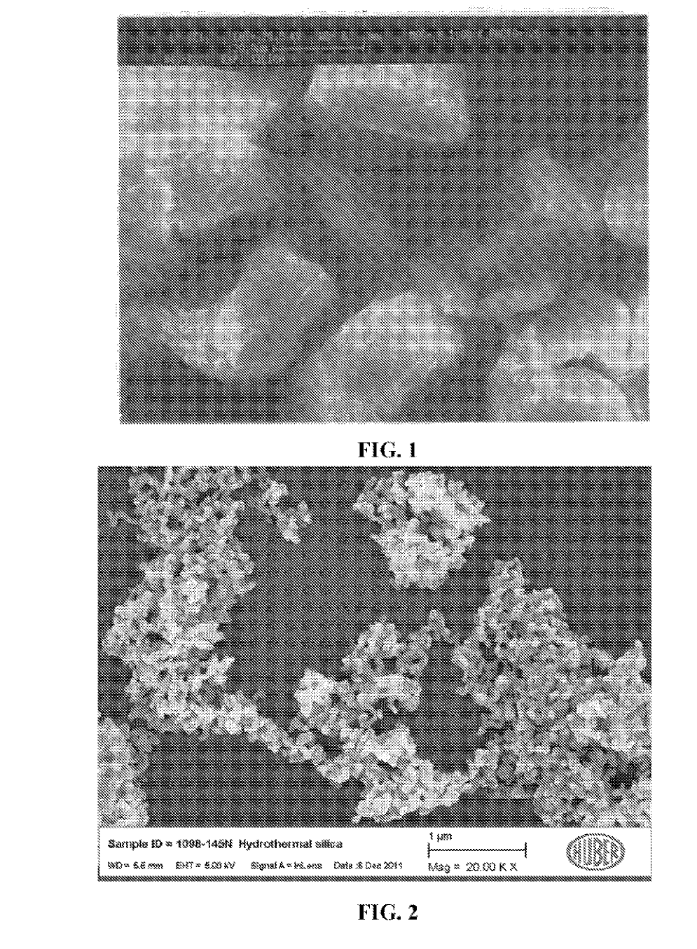

FIG. 1 illustrates a precursor material prior to hydrothermal treatment. FIG. 2 illustrates the resulting transformation of a similar silica material after hydrothermal treatment.

In one aspect, the hydrothermal process conditions (e.g., temperature, pressure, and time) are sufficient to produce a silica material, having a morphology of an agglomeration of dense, spherical or partially spherical particles, having high cleaning properties. It should be noted that if a sample is subjected to excessive hydrothermal conditions (e.g., temperature, pressure, pH or time), the silica material can crystallize or partially crystallize. In one aspect, such crystallinity can be undesirable. In one undesirable aspect, exposure to hydrothermal conditions for a period of about 10 hours (at 200° C.) can form crystalline or substantially crystalline materials. It should also be noted that the particular salt and concentration thereof can affect the crystallinity of the resulting material after exposure to hydrothermal conditions. The composition of a salt, if present, can also affect the level of crystallinity of a resulting silica material. In one aspect, a sulfate containing salt, such as, for example, sodium sulfate, can facilitate the formation of a desirable agglomerated silica material with high cleaning properties. In another aspect, the presence of a chloride containing salt, such, as sodium chloride, can result in the rapid formation of crystalline domains within the resulting silica material. For example, a precipitated silica heated to 220° C. in a saturated salt solution at pH 9.5 formed a crystalline silica material after 6 hours. As illustrated in FIG. 3, the material exhibited a crystalline morphology and was characterized as quartz. Allowing the silica material to form an incipient crystalline material, such as quartz can be undesirable. In a similar aspect, other crystalline forms of silica, such as cristobalite, can also be undesirable. Thus, in one aspect, the silica material does not comprise quartz. In another aspect, the silica material does not comprise magadiite, quartz, and/or cristobalite. In one aspect, the resulting silica material has a level of crystallinity equal to or approximately equal to that of a precipitated silica. In another aspect, the resulting silica material has a level of crystallinity of less than about 1%, or about 0.5%. In another aspect, the resulting silica material is amorphous or substantially amorphous, having at most only small levels (e.g., less than about 1% or about 0.5%) of crystallinity.

While not wishing to be bound by theory, it is believed that the surface area of the silica material can be reduced and particle size of the resulting silica particle agglomerates can be increased upon exposure to the elevated temperatures and optionally elevated pressures of a hydrothermal treatment step. In one aspect, the pH and the elevated temperature of the solution can result in a process wherein a portion of the silica is continuously dissolved and re-precipitated. In another aspect, the presence of the one or more salts or solutions thereof can improve this dissolution and precipitation, so as to form a silica material having desirable structure and morphological properties.

After hydrothermal treatment, the resulting material can be cooled and the pH optionally adjusted to a value of from between 7 and 8. The material can then be filtered and washed with, for example, distilled and/or distilled deionized water, and then dried. In one aspect, the material can be dried in an oven at about 105° C. for at least about 2 hours or overnight.

In one aspect, the method described herein comprises a two-step process, wherein silica is precipitated to a target pH value in a high salt concentration solution. The precipitated silica is then subjected to a hydrothermal treatment by heating the reaction slurry at elevated temperature to form a silica material having low surface area, high oil absorption number, and a morphology of an agglomerated spherical network of dense particles.

The inventive silica material described herein can, in one aspect, comprise a re-precipitated silica wherein at least a portion of the silica material has been rearranged through, for example, dissolution and precipitation occurring during the hydrothermal process. In one aspect, the inventive silica material, after hydrothermal treatment, does not comprise a silica gel. In another aspect, the inventive silica material, after hydrothermal treatment, does not comprise a calcined clay.

### Dentifrice Composition

The inventive silica materials can be ready-to-use additives in the preparation of oral cleaning compositions, such as dentifrices, toothpastes, and the like. In one aspect, the heat treated silica material can be combined with one or more dentifrice components, such as, for example, abrasives, rheological aids, whiteners, sweeteners, flavoring additives, surfactants, colorants, or other components to form a dentifrice composition. If combined with other abrasives (such as any of the products offered by J. M. Huber Corporation under the trade name ZEODENT®), such an abrasive may be added in any amount. In one aspect, the inventive silica material can be used at a loading of about 20 wt. % in the dentifrice composition. In other aspects, the inventive silica material can be used in excess of 20% and up to about 25 wt. %, 30 wt. %, 35 wt. % or more.

The inventive silica material can be utilized alone as the cleaning agent component in a dentifrice compositions or in combination with one or more other abrasive materials. Thus, a combination of the inventive materials with other abrasives physically blended therewith within a suitable dentifrice formulation can be useful to accord targeted dental cleaning and abrasion results at a desired protective level. Thus, any number of other conventional types of abrasive additives may be present within inventive dentifrices in accordance with this invention. Other such abrasive particles include, for example, and without limitation, precipitated calcium carbonate (PCC), ground calcium carbonate (GCC), dicalcium phosphate or its dihydrate forms, silica gel (and of any structure), amorphous precipitated silica (by itself, and of any structure as well), perlite, titanium dioxide, calcium pyrophosphate, hydrated alumina, calcined alumina, insoluble sodium metaphosphate, insoluble potassium metaphosphate, insoluble magnesium carbonate, zirconium silicate, aluminum silicate, and so forth, can be introduced within the desired abrasive compositions to tailor the polishing characteristics of the target formulation (dentifrices, for example, etc.), if desired, as well.

In addition, as noted above, the inventive silica material can be used in conjunction with other abrasive materials, such as precipitated silica, silica gel, dicalcium phosphate, dicalcium phosphate dihydrate, calcium metasilicate, calcium pyrophosphate, alumina, calcined alumina, aluminum silicate, precipitated and ground calcium carbonate, chalk, bentonite, particulate thermosetting resins and other suitable abrasive materials known to a person of ordinary skill in the art.

In addition to the abrasive component, a dentifrice can optionally comprise one or more organoleptic enhancing agents. Organoleptic enhancing agents include humectants, sweeteners, surfactants, flavorants, colorants and thickening agents, (also sometimes known as binders, gums, or stabilizing agents). Humectants serve to add body or “mouth texture” to a dentifrice as well as prevent the dentifrice from drying out. Suitable humectants can comprise polyethylene glycol (at a variety of different molecular weights), propylene glycol, glycerin (glycerol), erythritol, xylitol, sorbitol, mannitol, lactitol, and hydrogenated starch hydrolyzates, as well as mixtures of these compounds. Typical levels of humectants, if present, can range from about 20 wt % to about 30 wt % of a dentifrice composition.

Sweeteners can be added to a dentifrice composition to impart a pleasing taste to the product. Suitable sweeteners include saccharin (as sodium, potassium or calcium saccharin), cyclamate (as a sodium, potassium or calcium salt), acesulfane-K, thaumatin, neohisperidin dihydrochalcone, ammoniated glycyrrhizin, dextrose, levulose, sucrose, mannose, and glucose.

In one aspect, surfactants can also be used in a dentifrice composition to make the composition more cosmetically acceptable. A surfactant, if used, can be a detersive material which imparts to the composition detersive and foaming properties. Surfactants are safe and effective amounts of anionic, cationic, nonionic, zwitterionic, amphoteric and betaine surfactants such as sodium lauryl sulfate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine and the like can be used in a dentifrice together with the inventive silica material. A surfactant, if present, is typically used in an amount of about 0.1 to about 15% by weight, preferably about 0.3% to about 5% by weight, such as from about 0.3% to about 2%, by weight.

Flavoring agents optionally can be added to dentifrice compositions. Suitable flavoring agents include, but are not limited to, oil of wintergreen, oil of peppermint, oil of spearmint, oil of sassafras, and oil of clove, cinnamon, anethole, menthol, thymol, eugenol, eucalyptol, lemon, orange and other such flavor compounds to add fruit notes, spice notes, etc. These flavoring agents can comprise mixtures of aldehydes, ketones, esters, phenols, acids, and aliphatic, aromatic and other alcohols.

In addition, colorants can be added to improve the aesthetic appearance of the dentifrice product. Suitable colorants are selected from colorants approved by appropriate regulatory bodies such as the FDA and those listed in the European Food and Pharmaceutical Directives and include pigments, such as TiO₂, and colors such as FD&C and D&C dyes.

Thickening agents can, in various aspect, be useful in the dentifrice compositions of the present invention to provide a gelatinous structure that stabilizes the toothpaste against phase separation. In one aspect, the inventive high cleaning silica material exhibits thickening properties and would not require the addition of other thickener materials. In another aspect, one or more thickening agents can be used in addition to the high cleaning silica material. Suitable thickening agents include silica thickener; starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; as well as mixtures of these compounds. Typical levels of thickening agents or binders can range from about 0 wt % to about 15 wt % of a dentifrice composition. In one aspect, inventive silica material can impart thickening properties to a dentifrice composition, such that the dentifrice does not contain any additional thickening agents to provide desired rheological properties. In another aspect, the dentifrice does not contain any additional silica type thickening agents. In yet another aspect, the dentifrice does not comprise any thickening agents other than a cellulosic gum, such as, for example, sodium carboxymethylcellulose.

Therapeutic agents are optionally used in the compositions of the present invention to provide for the prevention and treatment of dental caries, periodontal disease and temperature sensitivity. Examples of therapeutic agents, without intending to be limiting, are fluoride sources, such as sodium fluoride, sodium monofluorophosphate, potassium monofluorophosphate, stannous fluoride, potassium fluoride, sodium fluorosilicate, ammonium fluorosilicate and the like; condensed phosphates such as tetrasodium pyrophosphate, tetrapotassium pyrophosphate, disodium dihydrogen pyrophosphate, trisodium monohydrogen pyrophosphate; tripolyphosphates, hexametaphosphates, trimetaphosphates and pyrophosphates, such as; antimicrobial agents such as triclosan, bisguanides, such as alexidine, chlorhexidine and chlorhexidine gluconate; enzymes such as papain, bromelain, glucoamylase, amylase, dextranase, mutanase, lipases, pectinase, tannase, and proteases; quaternary ammonium compounds, such as benzalkonium chloride (BZK), benzethonium chloride (BZT), cetylpyridinium chloride (CPC), and domiphen bromide; metal salts, such as zinc citrate, zinc chloride, and stannous fluoride; sanguinaria extract and sanguinarine; volatile oils, such as eucalyptol, menthol, thymol, and methyl salicylate; amine fluorides; peroxides and the like. Therapeutic agents can be used in dentifrice formulations singly or in combination at a therapeutically safe and effective level.

In another aspect, preservatives can also be optionally added to the compositions of the present invention to prevent bacterial growth. Suitable preservatives approved for use in oral compositions such as methylparaben, propylparaben and sodium benzoate, or combinations thereof, may be added in safe and effective amounts.

The dentifrices disclosed herein can also a variety of additional ingredients such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like. Water can be used in a dentifrice composition to balance the composition, for example, from about 0 wt. % to about 60 wt. %, and provide desirable rheological properties.

In yet another aspect, silica thickeners for use within a dentifrice composition can include, as a non-limiting example, an amorphous precipitated silica such as ZEODENT® 165 silica. Other silica thickeners can comprise ZEODENT® 163 and/or 167 and ZEOFREE® 153, 177, and/or 265 silicas, all available from J. M. Huber Corporation, Havre de Grace Md., U.S.A.

The invention described herein can be described in various non-limiting aspects, as recited below.

Aspect 1: A silica material comprising at least three of: a BET surface area of less than about 90 m²/g; an oil absorption number of at least about 80 cc/100 g; a loss on ignition of less than about 4 wt. %; and a PCR:RDA ratio of at least about 0.8 in a dentifrice comprising 11 wt. % glycerine (99.7%), 42.107 wt. % sorbitol (70%), 20 wt. % deionized water, 3 wt. % polyethylene glycol (PEG-12), 0.6 wt. % sodium carboxymethylcellulose, 0.5 wt. % tetrasodium pyrophosphate, 0.2 wt. % sodium saccharin, 0.243 wt. % sodium fluoride, 0.5 wt. % titanium dioxide, 1.2 wt. % sodium lauryl sulfate, 0.65 wt. % flavoring, and 20 wt. % of the silica material.

Aspect 2: The silica material of aspect 1, having each of a, b, c, and d.

Aspect 3: The silica material of aspect 1, being an agglomeration of dense spherical and/or partially spherical particles.

Aspect 4: The silica material of aspect 2, wherein the spherical and/or partially spherical particles have an average diameter of about 80 nm.

Aspect 5: The silica material of any preceding aspect, having a level of crystallinity of less than about 1%.

Aspect 6: The silica material of any preceding aspect, having a level of crystallinity of about 0.5% or less.

Aspect 7: The silica material of any preceding aspect, wherein the silica material does not comprise magadiite.

Aspect 8: The silica material of any preceding aspect, wherein the silica material is amorphous.

Aspect 9: The silica material of any preceding aspect, wherein the PCR is at least about 80 at a 20 wt. % loading of the silica material.

Aspect 10: The silica material of any preceding aspect, wherein the PCR is at least about 90 at a 20 wt. % loading of the silica material.

Aspect 12: The silica material of any preceding aspect, having a BET surface area of less than about 75 m²/g.

Aspect 13: The silica material of any preceding aspect, having a BET surface area of less than about 60 m²/g.

Aspect 14: The silica material of any preceding aspect, having a BET surface area of less than about 50 m²/g.

Aspect 15: The silica material of any preceding aspect, having a BET surface area of from about 15 m²/g to about 75 m²/g.

Aspect 16: The silica material of any preceding aspect, having a BET surface area of from about 15 m²/g to about 60 m²/g.

Aspect 17: The silica material of any preceding aspect, having a BET surface area of from about 15 m²/g to about 50 m²/g.

Aspect 18: The silica material of any preceding aspect, having an oil absorption number of at least about 90 cc/100 g.

Aspect 19: The silica material of any preceding aspect, having an oil absorption number of at least about 110 cc/100 g.

Aspect 20: The silica material of any preceding aspect, having an oil absorption number of at least about 120 cc/100 g.

Aspect 21: The silica material of any preceding aspect, having an oil absorption number of from about 90 cc/100 g to about 200 cc/100 g.

Aspect 22: The silica material of any preceding aspect, having an oil absorption number of from about 110 cc/100 g to about 200 cc/100 g.

Aspect 23: The silica material of any preceding aspect, having an oil absorption number of from about 120 cc/100 g to about 200 cc/100 g.

Aspect 24: The silica material of any preceding aspect, having a loss on ignition of less than about 3.5 wt. %.

Aspect 25: The silica material of any preceding aspect, having a loss on ignition of less than about 2.5 wt. %.

Aspect 26: The silica material of any preceding aspect, having a loss on ignition of from about 0.5 wt. % to about 4 wt. %.

Aspect 27: The silica material of any preceding aspect, having a loss on ignition of from about 0.5 wt. % to about 3.5 wt. %.

Aspect 28: The silica material of any preceding aspect, wherein the PCR:RDA ratio is at least about 0.9.

Aspect 29: The silica material of any preceding aspect, wherein the PCR:RDA ratio is at least about 1.

Aspect 30: The silica material of any preceding aspect, wherein the PCR:RDA ratio is from about 0.9 to about 1.5

Aspect 31: A dentifrice composition comprising the silica material of any preceding aspect.

Aspect 32: The dentifrice composition of aspect 31, not comprising any thickening agent other than the silica material or a cellulosic gum.

Aspect 33: The dentifrice composition of aspect 31, not comprising any thickening agent other than the silica material.

Aspect 34: A silica material comprising: a BET surface area of less than about 60 m²/g; an oil absorption number of at least about 120 cc/100 g; a loss on ignition of less than about 5 wt. %; and a PCR of at least about 80 and a PCR:RDA ratio of at least about 1.25 in a dentifrice comprising 11 wt. % glycerine (99.7%), 42.107 wt. % sorbitol (70%), 20 wt. % deionized water, 3 wt. % polyethylene glycol (PEG-12), 0.6 wt. % sodium carboxymethylcellulose, 0.5 wt. % tetrasodium pyrophosphate, 0.2 wt. % sodium saccharin, 0.243 wt. % sodium fluoride, 0.5 wt. % titanium dioxide, 1.2 wt. % sodium lauryl sulfate, 0.65 wt. % flavoring, and 20 wt. % of the silica material.

Aspect 35: A method for a silica material, the method comprising: subjecting a precursor material to a hydrothermal process.

Aspect 36: The method of aspect 35, wherein the precursor material is prepared by contacting a silicate compound or a solution thereof with an acid in the presence of a salt or solution thereof, so as to form the precursor material.

Aspect 37: The method of aspect 36, wherein the silicate compound comprises a metal:silicate molar ratio of from about 1:1 to about 1:3.5.

Aspect 38: The method of aspect 35 or 36, wherein the hydrothermal conditions comprise heating to a temperature of from about 160° C. to about 220° C. for a period of from about 1.5 to about 2 hours.

Aspect 39: The method of aspect 35 or 36, wherein the hydrothermal conditions comprise a pressure of from about 150 psi to about 300 psi.

Aspect 40: The method of aspect 35 or 36, wherein the hydrothermal conditions do not form magadiite.

Aspect 41: The method of aspect 36, wherein the salt or solution thereof comprises sodium sulfate.

Aspect 42: The method of aspect 36, wherein the salt is present at a concentration of from about 0.1 g to about 30 g per 100 g of water.

Aspect 43: The method of aspect 36, wherein the salt or solution thereof comprises a 10 wt. % solution of sodium sulfate.

Aspect 44: The method of aspect 36, wherein the silicate compound or solution thereof is present at a concentration of from about 8 wt. % to about 35 wt. %.

Aspect 45: A silica material formed by the method of any of aspects 35-44.

Aspect 46: The silica material of aspect 45, having at least three of: a BET surface area of less than about 90 m²/g; an oil absorption number of at least about 80 cc/100 g; a loss on ignition of less than about 4 wt. %; and a PCR:RDA ratio of at least about 0.8 in a dentifrice comprising 11 wt. % glycerine (99.7%), 42.107 wt. % sorbitol (70%), 20 wt. % deionized water, 3 wt. % polyethylene glycol (PEG-12), 0.6 wt. % sodium carboxymethylcellulose, 0.5 wt. % tetrasodium pyrophosphate, 0.2 wt. % sodium saccharin, 0.243 wt. % sodium fluoride, 0.5 wt. % titanium dioxide, 1.2 wt. % sodium lauryl sulfate, 0.65 wt. % flavoring, and 20 wt. % of the silica material.

Aspect 47: The silica material of aspect 46, having each of a, b, c, and d.

Aspect 48: The silica material of aspect 45, being an agglomeration of dense spherical and/or partially spherical particles.

Aspect 49: The silica material of aspect 45, wherein the spherical and/or partially spherical particles have an average diameter of about 80 nm.

Aspect 50: The silica material of any of aspects 45-49, having a level of crystallinity of less than about 1%.

Aspect 51: The silica material of any of aspects 45-50, having a level of crystallinity of about 0.5% or less.

Aspect 52: The silica material of any of aspects 45-51, wherein the silica material does not comprise magadiite.

Aspect 53: The silica material of any of aspects 45-52, wherein the PCR is at least about 80 at a 20 wt. % loading of the silica material.

Aspect 54: The silica material of any of aspects 45-53, wherein the PCR is at least about 90 at a 20 wt. % loading of the silica material.

Aspect 55: The silica material of any of aspects 45-54, having a hydroxyl group density of less than about 2%.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1—Preparation of Inventive High Cleaning Silica Materials

In a first example, high cleaning silica materials were prepared by adding 2,000 mls of water to a Parr bomb and stirring at 400 rpm. Then, 560 grams of sodium sulfate and 650 mls of 17.1% sulfuric acid were added to the Parr bomb, together with an appropriate volume (e.g., 1,200 mls) of 3.3 MR 24.7% sodium silicate at a rate of 60 mls/min until a pH of 8.0 was reached.

The stir rate was increased as the solution began to gel. 1,000 mls of water was then added as the stir rate was increased to 900 rpm. The solution was stirred for another 15 minutes, and sodium silicate was added as necessary to maintain a pH of 8.0. The Parr bomb was then assembled and heated to 220° C. for 2 hours while stirring at 400 rpm. The internal pressure of the Parr bomb was 290 psi. After 2 hours at 220° C., the bomb was cooled and any residual pressure was bled off.

The resulting material was removed from the Parr bomb, filtered, and washed three times with water. The washed material was then dried in an oven at 105° C. overnight.

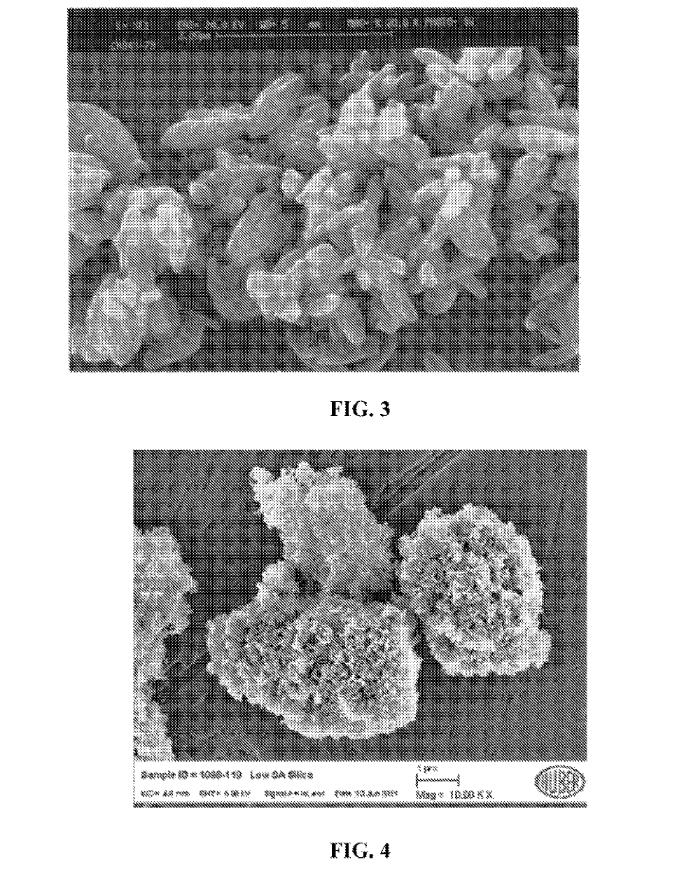

FIGS. 4 and 5 are scanning electron micrographs of silica materials produced using the method recited in this Example. Table 1, below, details analytical properties of the resulting silica material. PCR and RDA values were obtained on a dentifrice comprising 11 wt. % glycerine (e.g, 99.7%), 42.107 wt. % sorbitol (e.g., 70%), 20 wt. % deionized water, 3 wt. % polyethylene glycol (e.g., PEG-12), 0.6 wt. % sodium carboxymethylcellulose (e.g., Cekol 2000, available from CP Kelco US, Inc.), 0.5 wt. % tetrasodium pyrophosphate, 0.2 wt. % sodium saccharin, 0.243 wt. % sodium fluoride, 0.5 wt. % titanium dioxide, 1.2 wt. % sodium lauryl sulfate, 0.65 wt. % flavoring, and 20 wt. % of the silica material.

TABLE 1
Properties of High Cleaning Silica Materials produced in
Example 1

| Property | Value | |
|---|---|---|
| Surface Area | | |
| CTAB | 41 | m²/g |
| BET | 32 | m²/g |
| Oil Absorption Number | 118 cc/100 g | |
| 5% pH | 9.63 | |
| Loss on Ignition | 1.67 | wt. % |
| PCR (20% loading) | 113 | |
| RDA (20% loading) | 122 | |
| XRD | Amorphous | |
| Einlehner Abrasion | 12.5 | mg lost |
| Total Sulfate (LECO) | 0.55 | wt. % |
| Horiba P/S Med (2 min/U/S) | 13.04 | μm |

### Example 2—Preparation of Inventive Silica Materials (Large Batch)

In a second example, high cleaning silica materials were prepared on an intermediate scale by adding 300 liters of water to a reactor at ambient conditions and stirring at 80 rpm. 84 kg of sodium sulfate (e.g., to provide a 10 wt. % solution based on the total front heel volume) was then added to the reactor, followed by 300 liters of 2.5 MR 20.0% sodium silicate. Sulfuric acid (17.1%) was then added at a rate of 4.35 liters/min until the pH reached 8.5. After the addition of about 72 liters of sulfuric acid (i.e., at about pH 10.51, 16.37 minutes), the material exhibited opalescent behavior. When the solution gels during acid addition, an additional 150 liters of water as added. Once pH 8.5 was reached, the solution was stirred while digesting for 10 minutes. At the end of the digestion period, the pH was readjusted with sodium silicate to maintain a pH of 8.5.

The reaction slurry was then pumped into a pressure reactor, heated to 190° C., and held at 190° C. for two hours while stirring. The pressure in the reactor was 160 psi. After two hours, the reactor was depressurized and the resulting material transferred to a drop tank. The pH was adjusted to 7.5 using 17.1% sulfuric acid. The resulting material was then filtered and washed with water to a conductivity OF 2,000 μs and then spray dried.

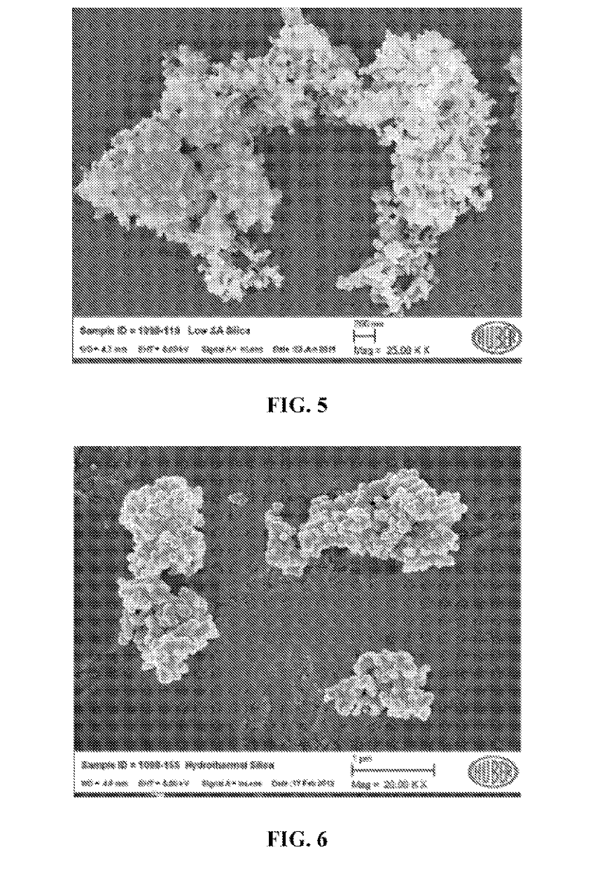

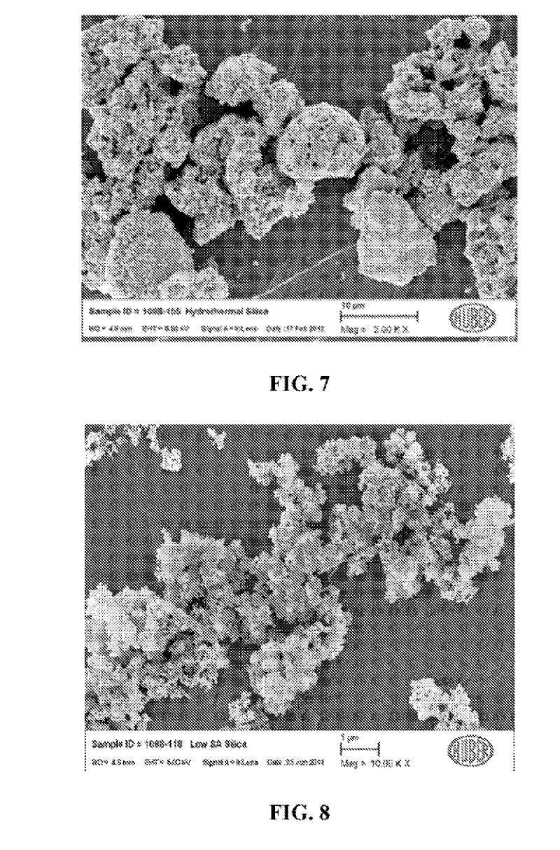

FIGS. 6 and 7 are scanning electron micrographs of silica materials produced using the method recited in this Example. Table 2, below, details analytical properties of the resulting silica material. PCR and RDA values were obtained on a dentifrice comprising 11 wt. % glycerine (e.g, 99.7%), 42.107 wt. % sorbitol (e.g., 70%), 20 wt. % deionized water, 3 wt. % polyethylene glycol (e.g., PEG-12), carboxymethylcellulose (e.g., Cekol 2000, available from CP Kelco US, Inc.), 0.5 wt. % tetrasodium pyrophosphate, 0.2 wt. % sodium saccharin, 0.243 wt. % sodium fluoride, 0.5 wt. % titanium dioxide, 1.2 wt. % sodium lauryl sulfate, 0.65 wt. % flavoring, and 20 wt. % of the silica material

TABLE 2
Properties of High Cleaning Silica Materials produced in
Example 2

| Property | Value | |
|---|---|---|
| Surface Area | | |
| CTAB | 50 | m²/g |
| BET | 51 | m²/g |
| Oil Absorption Number | 164 cc/100 g | |
| 5% pH | 8.50 | |
| Loss on Ignition | 4.81 | wt. % |
| PCR (20% loading) | 86 | |
| RDA (20% loading) | 60 | |
| XRD | Amorphous | |
| Einlehner Abrasion | 3.01 | mg lost |
| Median Particle Size | 18.3 | μm |
| Technidyne Brightness | 100.1 | |
| Sodium sulfate by conductivity | 1.37 | wt. % |

| | |
|---|---|
| Water AbC corrected | 234 |

Refractive index (n) and transmission (% T) measurements were also performed on glycerin and sorbitol solutions containing the silica material (i.e., 10 wt. %) prepared in this Example, as detailed below.

TABLE 3
Refractive Index and Sorbitol Measurements

| Solution | n | % T |
|---|---|---|
| Glycerol, 85% | 1.451 | 14.3 |
| 87% | 1.454 | 71.0 |
| 89% | 1.457 | 78.4 |
| 91% | 1.460 | 76.4 |
| 93% | 1.463 | 59.2 |
| Sorbitol, 59% | 1.435 | 21.2 |
| 61% | 1.439 | 32.1 |
| 63% | 1.443 | 47.0 |
| 65% | 1.447 | 60.3 |
| 67% | 1.451 | 71.8 |
| 69% | 1.455 | 79.9 |

### Example 3—Preparation of Inventive Silica Materials

In a third example, high cleaning silica materials were prepared by adding 2,000 mls of water to a Parr bomb and stirring at 400 rpm. 560 grams of sodium sulfate and 1,600 mls of 3.3 MR 24.& % sodium silicate were then added to the water in the Parr bomb. Sulfuric acid (17.1 wt. %) was then added to the resulting solution at a rate of 30 mls/min until a final pH of 8.0 was reached. As the solution began to gel, the stirring speed was increased and an additional 1,000 mls of water was added until the stir speed reached 900 rpm. Once the pH reached 8.0, stirring was continued for an additional 15 minutes and the pH was readjusted to maintain a value of 8.0. The Parr bomb was assembled, heated to 220° C., and maintained at 220° C. for 2 hours while stirring at 400 rpm. The Parr bomb was then cooled and the residual pressure bled off. The resulting material was filtered and washed three times with water before drying overnight in an oven at 105° C.

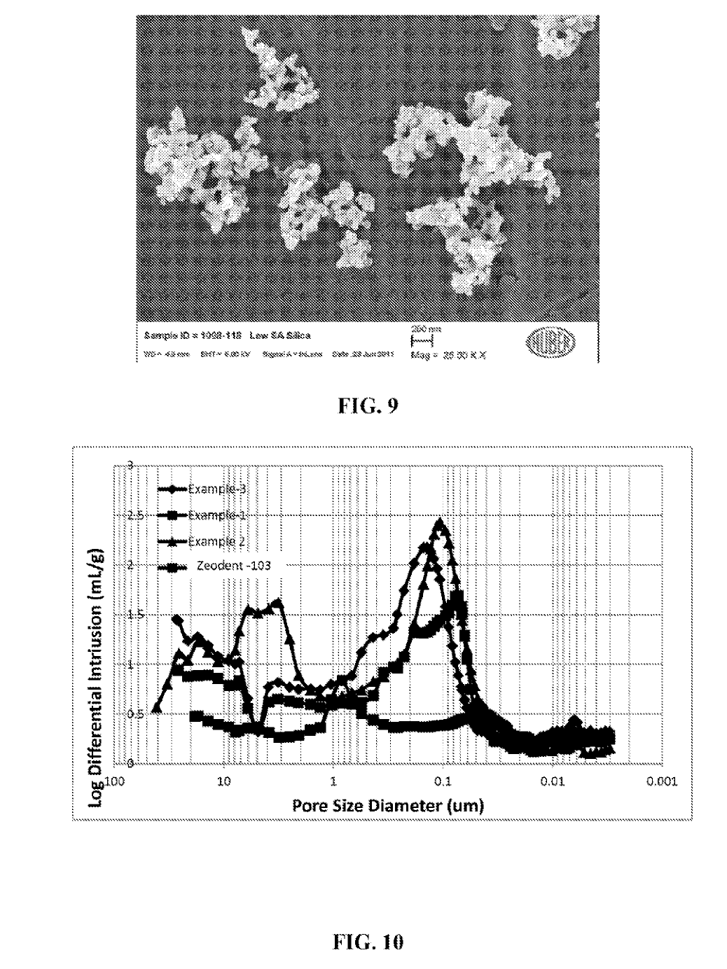

FIGS. 8 and 9 are scanning electron micrographs of the resulting high cleaning silica material prepared from this Example.

TABLE 4
Properties of High Cleaning Silica Materials produced in
Example 3

| Property | Value | |
|---|---|---|
| CTAB Surface Area | 34 | m²/g |
| BET Surface Area | 29 | m²/g |
| Oil Absorption Number | 183 cc/100 g | |
| 5% pH | 9.63 | |
| Loss on Ignition | 2.18 | wt. % |
| XRD | Amorphous | |
| Einlehner Abrasion | 8.0 | mg lost |
| Total Sulfate (LECO) | 0.73 | wt. % |
| Median Particle Size | 12.01 | μm |

### Example 4—Mercury Porosimetry

In a fourth example, samples of the high cleaning silica materials prepared in Examples 1 and 3 were compared to a conventional Zeodent®-103 silica, available from J. M. Huber, Atlanta, Ga., USA. Zeodent®-103 silica typically has an Oil Absorption Number of from 50 to 65, an average particle size of 7 μm to 11 μm, a BET surface area of about 50 m²/g, and a PCR:RDA ratio of from about 0.45 to about 0.56. While the inventive silica materials described herein exhibit lower surface areas than Zeodent®-103, the exhibit oil absorption values 2-3 times higher than that of the Zeodent®-103.

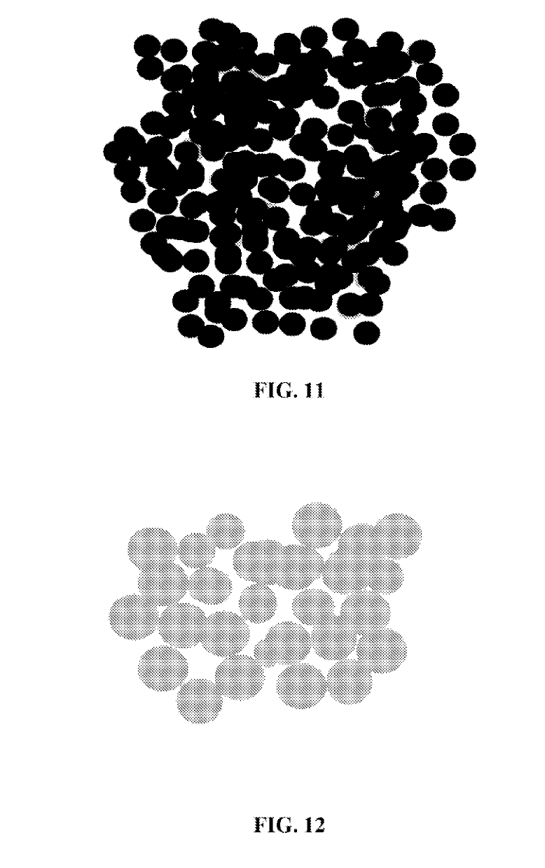

As evidenced by the mercury porosimetry data illustrated in FIG. 10, the inventive silica materials described herein (“EX. 1”, “EX. 3”) exhibit a greater number of inter-particle void spaces, capable of absorbing oil, than the tightly packed dense particles of Zeodent®-103 (“Z-103”). FIG. 11 is a schematic illustration of the tightly packed dense silica particles of Zeodent®-103. In contrast, FIG. 12 is a schematic illustration of the larger dense particles of the inventive high cleaning silica materials having larger void spaces.

### Example 5—Viscosity Measurements

In a fifth example, the thickening properties of the inventive silica was evaluated by measuring the viscosity a sample of the silica material prepared in Example 3 to two conventional Zeodent® silica materials, Zeodent®-165 (“Z-165”) and Zeodent®-153 (“Z-153”).

The build in viscosity was measured for each material after 24 hours, 1 week, 3, weeks, 6 weeks, and 9 weeks, as detailed below. Toothpaste formulations comprising the silica materials were prepared. Viscosity measurements were then performed using a Brookfield viscometer. FIG. 13 illustrates the build in viscosity for each of the three silica materials over a period of time. As noted above, the inventive silica materials can exhibit desirable thickening properties in a dentifrice without the need for additional thickening agents.

TABLE 5
Viscosity Build Data for Silica Materials

| | | EX. 3 | Z-165 | Z-153 |
|---|---|---|---|---|
| | | Viscosity (centipoises) | | |
| 24 | Hours | 70,000 | 159,000 | 72,000 |
| 1 | Week | 102,000 | 209,000 | 107,000 |
| 3 | Weeks | 125,000 | 260,000 | 131,000 |
| 6 | Weeks | 136,000 | 281,000 | 143,000 |
| 9 | Weeks | 148,000 | 299,000 | 150,000 |

While significant differences exist in the structure and surface area values between the inventive high cleaning silica and the conventional silica thickener Zeodent®-153, both materials exhibit similar viscosity build over time, as detailed in Table 5, above, and in FIG. 13. The inventive silica material is thus suitable for use in dentifrice and oral care compositions.

### Example 6—Crystallinity of Resulting Silica Material

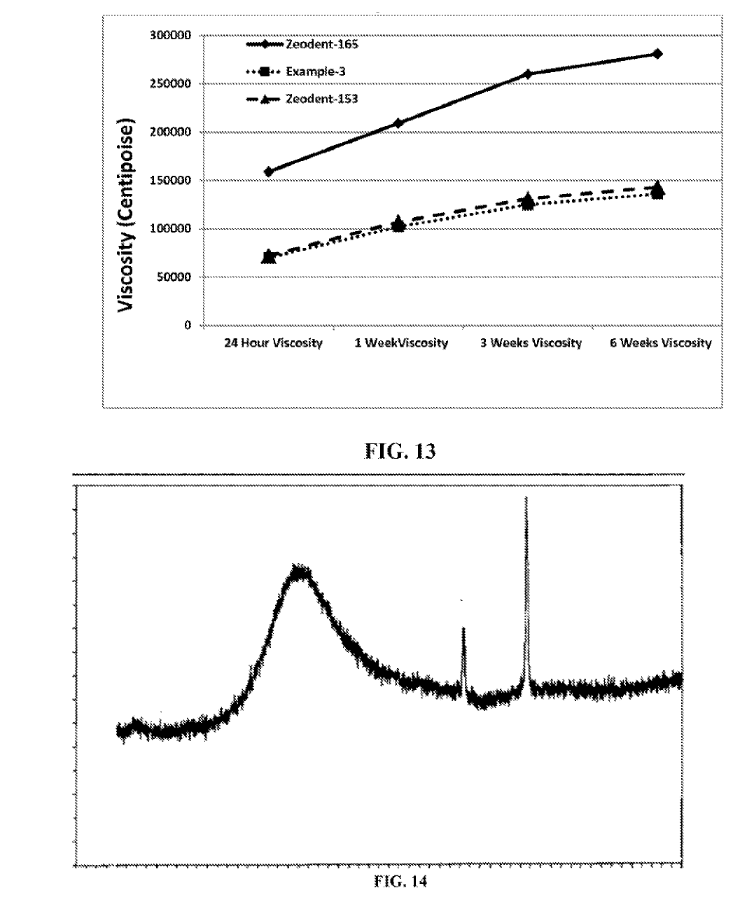

As described above, the inventive silica material is, in one aspect, an amorphous material having little or no crystallinity. As illustrated in the x-ray diffraction pattern of FIG. 14, the inventive silica material is amorphous. The only peaks appearing in the XRD pattern were the result of the aluminum sample holder used in the experiment.

In one aspect, a silica or silicate material subjected to excessive hydrothermal conditions can produce a crystalline material, such as, for example, magadiite or quartz. Two samples were prepared, each by adding 500 mls of water to a Parr bomb and stirring at 400 rpm. To each, 420 g of sodium sulfate was added to the water, and then 400 mls of 3.3 MR 24.7% sodium silicate was added to the Parr bomb reactor, followed by an appropriate volume (e.g., about 245 mls) of acid at a rate of 30 mls/min to obtain a pH of 9.5. The stirring was then increased as the solutions began to gel, at which time 250 mls of water was added while increasing the stir rate to 800 rpm. At pH 9.5, stirring was continued for an additional 15 minutes while continuously readjusting the pH to maintain a value of 9.5.

The Parr bomb for the first sample (Sample X) was assembled and heated to 220° C. while stirring at 400 rpm, resulting in an internal pressure of 370 psi. Once the temperature reached 220° C., the temperature was maintained for two hours, after which the Parr bomb was cooled and any residual pressure bled off. The resulting material was filtered and washed three times with water, and then oven dried at 105° C. overnight.

The Parr bomb for the second sample (Sample Y) was assembled and heated to 220° C. while stirring at 400 rpm, resulting in an internal pressure of 370 psi. Once the temperature reached 220° C., the temperature was maintained for six hours, after which the Parr bomb was cooled and any residual pressure bled off. The resulting material was filtered and washed three times with water, and then oven dried at 105° C. overnight. The properties of the resulting materials are detailed in Table 6, below.

TABLE 6
Properties of Silica Materials prepared at Increased Hydrothermal
Conditions

| | Sample X | | Sample Y | |
|---|---|---|---|---|
| CTAB Surface Area | 195 | m2/g | 11 | m2/g |
| BET Surface Area | 24 | m2/g | 9 | m2/g |
| Oil Absorption Number | 129 cc/100 g | | 54 cc/100 g | |
| Median Particle Size | 3.08 | μm | 0.82 | μm |

| | | |
|---|---|---|
| Crystallinity | Magadiite | Quartz |

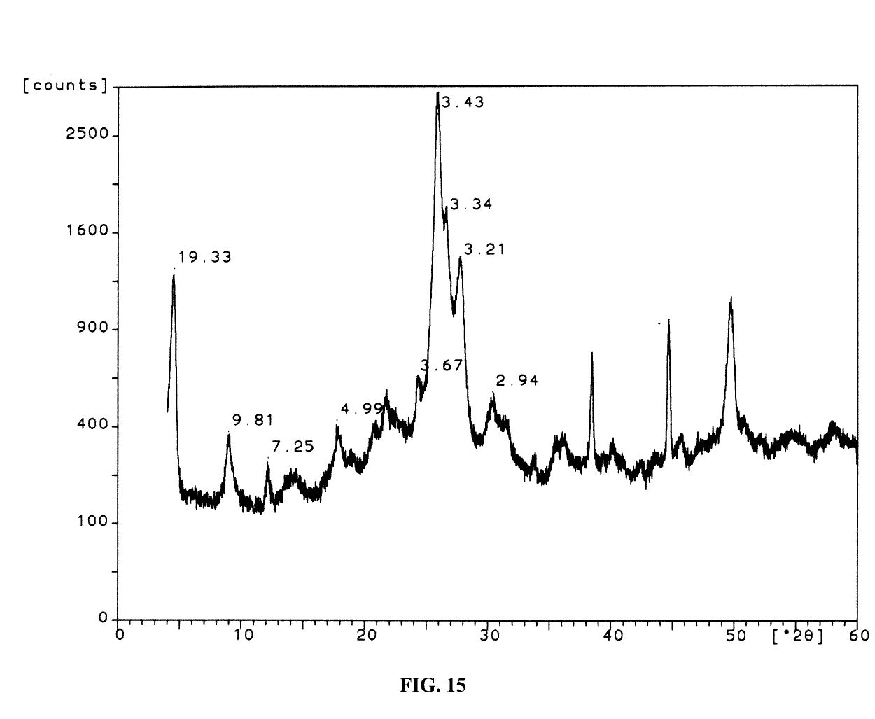

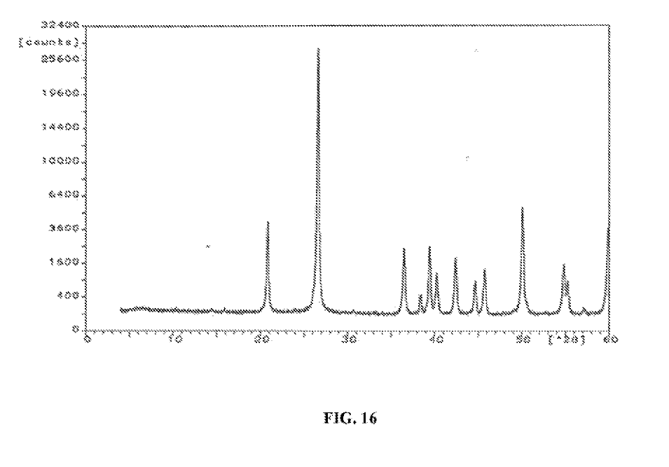

X-ray diffraction patterns for magadiite and quartz are illustrated in FIGS. 15 and 16, respectively. The x-ray diffraction patterns for these materials illustrate their crystalline nature, in contrast to the pattern in FIG. 14 of an amorphous silica material.

1-14. (canceled)

15. A method for preparing a silica material, the method comprising:
a. contacting a silicate compound or a solution thereof with an acid in the presence of a salt or solution thereof, to form a precursor material, and
b. subjecting the precursor material to hydrothermal conditions.

16. The method of claim 15, wherein the silicate compound comprises a metal:silicate molar ratio of from about 1:1 to about 1:3.5.

17. The method of claim 15, wherein the hydrothermal conditions comprise heating to a temperature of from about 160° C. to about 220° C. for a period of from about 1.5 to about 2 hours.

18. The method of claim 15, wherein the hydrothermal conditions comprise a pressure of from about 150 psi to about 300 psi.

19-20. (canceled)

21. The method of claim 15, wherein the silicate compound comprises sodium silicate.

22. The method of claim 15, wherein the salt comprises sodium sulfate, sodium phosphate, sodium chloride, or a combination thereof.

23. The method of claim 15, wherein the acid comprises nitric acid, hydrochloric acid, phosphoric acid, boric acid, hydrofluoric acid, sulfuric acid, or a combination thereof.

24. The method of claim 15, wherein the acid comprises sulfuric acid.

25. The method of claim 15, wherein the silica material comprises silica.

26. The method of claim 15, wherein the silica material comprises sodium aluminosilicate.

27. The method of claim 15, wherein the hydrothermal conditions comprise a temperature of from about 100° C. to about 350° C. in a sealed vessel.

28. The method of claim 15, wherein the hydrothermal conditions comprise a pressure of from about 150 psi to about 300 psi and a temperature of from about 120° C. to about 240° C. in a sealed vessel.

29. The method of claim 15, wherein the method further comprises, after step b, a pH adjusting step, a filtering step, a washing step, a drying step, or any combination thereof, to form the silica material.

30. The method of claim 29, wherein the BET surface area of the silica material is less than that of the precursor material.

31. The method of claim 29, wherein the silica material comprises an agglomeration of dense spherical and/or partially spherical particles.

32. The method of claim 31, wherein the spherical and/or partially spherical particles have an average diameter of about 80 nm.

33. The method of claim 29, wherein the silica material has a level of crystallinity of less than about 1%.

34. The method of claim 29, wherein the silica material is characterized by:
(a) a BET surface area of from about 5 m²/g to about 45 m²/g;
(b) an oil absorption number of at least about 80 cc/100 g;
(c) a loss on ignition of less than about 4 wt. %; and
(d) a PCR:RDA ratio of at least about 0.8 in a dentifrice comprising 11 wt. % glycerine (99.7%), 42.107 wt. % sorbitol (70%), 20 wt. % deionized water, 3 wt. % polyethylene glycol (PEG-12), 0.6 wt. % sodium carboxymethylcellulose, 0.5 wt. % tetrasodium pyrophosphate, 0.2 wt. % sodium saccharin, 0.243 wt. % sodium fluoride, 0.5 wt. % titanium dioxide, 1.2 wt. % sodium lauryl sulfate, 0.65 wt. % flavoring, and 20 wt. % of the silica material.

35. The method of claim 15, wherein:
the silicate compound comprises sodium silicate;
the salt comprises sodium sulfate;
the acid comprises sulfuric acid; and
the hydrothermal conditions comprise a temperature of from about 100° C. to about 350° C. in a sealed vessel.

36. The method of claim 35, wherein the method further comprises, after step b, a filtering step, a washing step, and a drying step, to form the silica material.

37. The method of claim 36, wherein:
the BET surface area of the silica material is less than that of the precursor material; and
the median particle size of the silica material is greater than that of the precursor material.
